(19) United States
(12) Patent Application Publication
(10) Pub. No.: US 2011/0060209 A1
(43) Pub. Date: Mar. 10, 2011

(54) INTRACORPOREAL MONITORING APPARATUS HAVING FLECTION

(75) Inventors: Hiroyuki SHIKATA, Nasushiobara-shi (JP); Hideki KOSAKU, Nasushiobara-shi (JP); Takashi TAKEUCHI, Otawara-shi (JP)

(73) Assignees: Kabushiki Kaisha Toshiba, Tokyo (JP); TOSHIBA MEDICAL SYSTEMS CORPORATION, Otawara-shi (JP)

(21) Appl. No.: 12/873,607

(22) Filed: Sep. 1, 2010

(30) Foreign Application Priority Data
Sep. 7, 2009 (JP) 2009-206095

(51) Int. Cl. A61B 5/05

(52) U.S. Cl. 600/407

(57) ABSTRACT

In one embodiment, an intracorporeal monitoring apparatus having a flection includes: a tip unit that receives an ultrasonic signal or light from inside a subject and converts the ultrasonic signal or light into an electric signal; a guiding unit that transmits the electric signal obtained by the tip unit; and a flection that is provided between the guiding unit and the tip unit, transmits the electric signal obtained by the tip unit to the guiding unit, and can be bent, the flection has a conductor that is curved and extensible, one or a plurality of layers of conductive tubes coating the conductor, and a layer of an insulating tube coating the conductive tubes.

### CROSS-REFERENCE TO RELATED APPLICATION

This application is based upon and claims the benefit of priority from Japanese Patent Application No. 2009-206095, filed on Sep. 7, 2009, the entire contents of which are incorporated herein by reference.

### FIELD

Embodiments of the present invention relate to an intracorporeal monitoring apparatus having a flection for ultrasonic diagnosis such as a transesophageal echocardiography probe, endoscope, and ultrasonic laparoscope.

### BACKGROUND

A transesophageal echocardiography (TEE) probe is an ultrasonic probe to diagnose the heart through esophageal walls or stomach walls after being perorally inserted into the esophagus. The TEE probe includes a tip unit that transmits/receives an ultrasonic wave, a guiding unit to insert the TEE probe into the esophagus, a flection that connects the guiding unit and the tip unit and whose angle of bend is operable, an operation unit that operates the angle of bend of the flection, and a connector unit to connect the TEE probe to the body of ultrasonic diagnostic equipment.

The flection normally has, like an endoscope for a digestive organ, a plurality of metallic bending mechanisms linked by a link mechanism coated with an angle tube. Elasticity of the angle tube determines the degree of bendability of the flection.

Rubber material superior in durability and biocompatibility such as fluorubber and silicon rubber is generally used for the angle tube. To prevent an unintentional current from being passed to a patient from an external power supply, it is necessary for the guiding unit, the flection, and the tip unit that come into contact with the patient to adopt an F-type applied portion to be electrically floating. Thus, also the angle tube preferably has insulating properties.

A flection that can be bent as flexibly as possible is demanded for an endoscope for a digestive organ to improve operability thereof in the stomach or duodenum. On the other hand, a TEE probe does not normally have an optical system and thus, the laryngopharynx, esophagus, gastric mucosa and the like may be damaged depending on how the probe is inserted or operated. Therefore, flexing resistance properties that do not allow the flection to bend easily are demanded to prevent the tip unit from being abruptly bent when the TEE probe is inserted or operated.

There are mainly two methods to realize such flexing resistance properties. One method is to enhance flexing resistance properties of the flection itself by changing the elastic constant of the angle tube used for the flection. The other method is to attach a mechanism to a control knob that operates the flection so that the flection is not easily bent.

For the former method of improving flexing resistance properties by changing the elastic constant of the angle tube, rubber material that does not stretch much is selected and used from the start or the rubber is made thicker to be able to enhance the restoring force or repulsive force.

However, if the angle rubber of a TEE probe is made thicker, the rubber will not stretch so that the angle tube cannot be passed through the tip unit having a larger diameter than the guiding unit during assembly or repairs/replacement. Thus, depending on the stretch amount of the angle tube, the diameter of the tip unit may be limited.

Making the tip unit still smaller can reduce a physical burden of the patient during insertion because smooth movement of the tip unit is thereby enabled inside a body cavity, but there is a problem that image quality deteriorates due to a smaller aperture of ultrasonic transducers. Thus, to make replacement of the angle tube easier while giving the same elastic constant to the angle rubber, for example, a method of doubling the angle tube with half a thickness each to make each angle tube easier to stretch and replacement thereof easier can be considered.

Though not intended to make replacement of the angle tube easier, an example of doubling the angle tube in a flection is known in an endoscope for the purpose of detecting an occurrence of angle tube defects (see, for example, Japanese Patent Application Laid-Open No. 2005-211432).

The control knob can be made not to rotate easily by a friction mechanism or a rotary click mechanism as a mechanism that does not allow the flection to bend easily. However, a problem of long-term stability or a problem of causing the cost to rise due to more complex mechanisms may arise.

The angle tube may be bitten by the patient while being used, or fractured due to an external force while being handled or ageing. If the angle tube is fractured and a hole is cut, a problem of the inner mechanism being affected by a liquid breaking into the probe or of becoming a source of infection after bacteria being propagated in the hole may arise.

An endoscope disclosed by Japanese Patent Application Laid-Open No. 2005-211432 has a structure in which a gas can be infused into a space between rubber members of the double angle tubes so that a break in the angle tubes can easily be detected.

A TEE probe having no structure allowing a gas to infuse into, on the other hand, has a mechanism to detect presence/absence of a hole by soaking the tip unit in water in the inspection before the use to measure insulation between water around the tip unit and metal inside the probe. If the insulating angle tube is doubled in such a TEE probe, insulation properties are maintained even if a hole is cut in outer rubber so that an occurrence of the defect cannot be detected. Then, there is a danger of infection among patients after bacteria breaking into a space between the outer rubber and inner rubber from the hole in the outer rubber.

In the foregoing, only a TEE probe has been described, but an endoscope and an ultrasonic laparoscope also have a flection and similar problems also arise in each flection.

The present invention provides an intracorporeal monitoring apparatus having a flection in which an occurrence of defects in the angle tube can easily be detected and also the angle tube can easily be replaced.

### BRIEF DESCRIPTION OF THE DRAWINGS

FIG. 1 is a schematic view of a transesophageal echocardiography probe according to an embodiment.

FIG. 2 is a diagram showing an internal structure of a flection of the transesophageal echocardiography probe according to an embodiment.

FIG. 3 is a diagram showing a sectional structure of the flection of the transesophageal echocardiography probe according to an embodiment.

FIG. 4 is a diagram illustrating an apparatus to inspect whether there is a hole in the flection in the present embodiment.

FIG. 5 is a perspective view showing the structure of an ultrasonic laparoscope in another embodiment.

FIG. 6 is a perspective view showing the structure of an endoscope in still another embodiment.

### DETAILED DESCRIPTION

According to an embodiment of the present invention, an intracorporeal monitoring apparatus having a flection, includes

a tip unit that receives an ultrasonic signal or light from inside a subject and converts the ultrasonic signal or light into an electric signal;

a guiding unit that transmits the electric signal obtained by the tip unit; and

a flection that is provided between the guiding unit and the tip unit, transmits the electric signal obtained by the tip unit to the guiding unit, and can be bent, the flection has a conductor that is curved and extensible, one or a plurality of layers of conductive tubes coating the conductor, and a layer of an insulating tube coating the conductive tubes.

According to an embodiment of the present invention, an occurrence of an angle tube defect can be detected so that an intracorporeal monitoring apparatus having a flection in which the angle tube can easily be replaced and flexing properties of the flection are improved can be obtained.

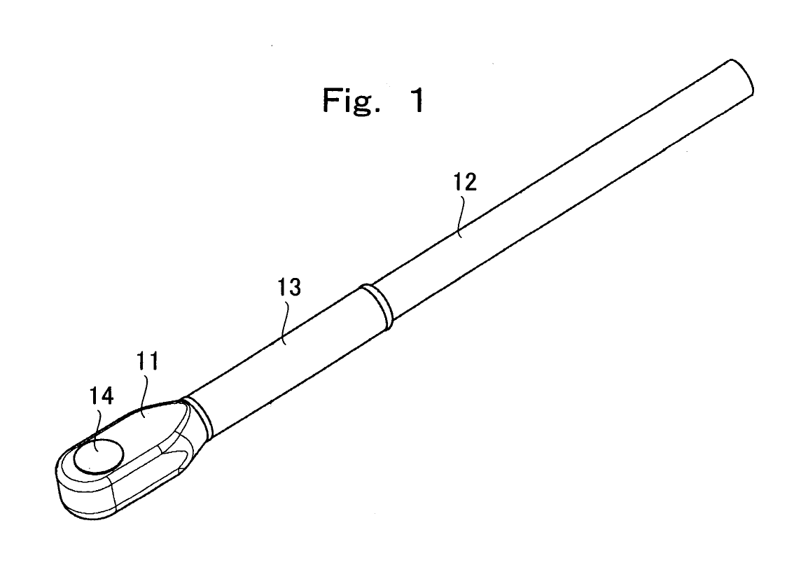

An embodiment of the present invention will be described in detail with reference to drawings. FIG. 1 is a schematic view of a transesophageal echocardiography (TEE) probe according to an embodiment. The TEE probe includes a tip unit 11 that transmits/receives an ultrasonic wave into/from a subject, a guiding unit 12 used for insertion into the esophagus, and a flection 13 that connects the tip unit 11 and the guiding unit 12 and whose angle of bend is operable.

Though not illustrated, the TEE probe further includes an operation unit that can operate the angle of bend of the flection 13 and a connector unit to connect to the body of ultrasonic diagnostic equipment. The tip unit 11 contains ultrasonic transducers 14, which transmit/receive an ultrasonic wave. The flection 13 has angle links described later coated with an angle tube therearound.

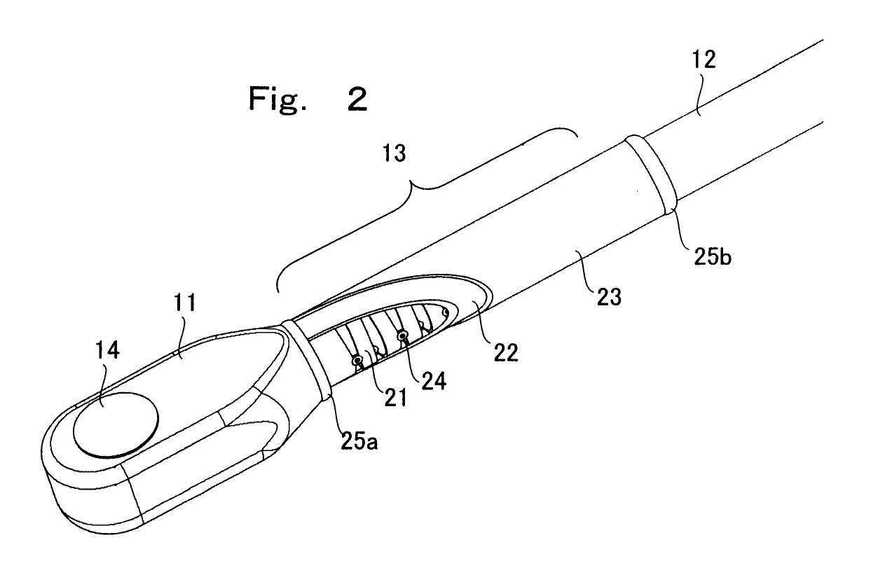

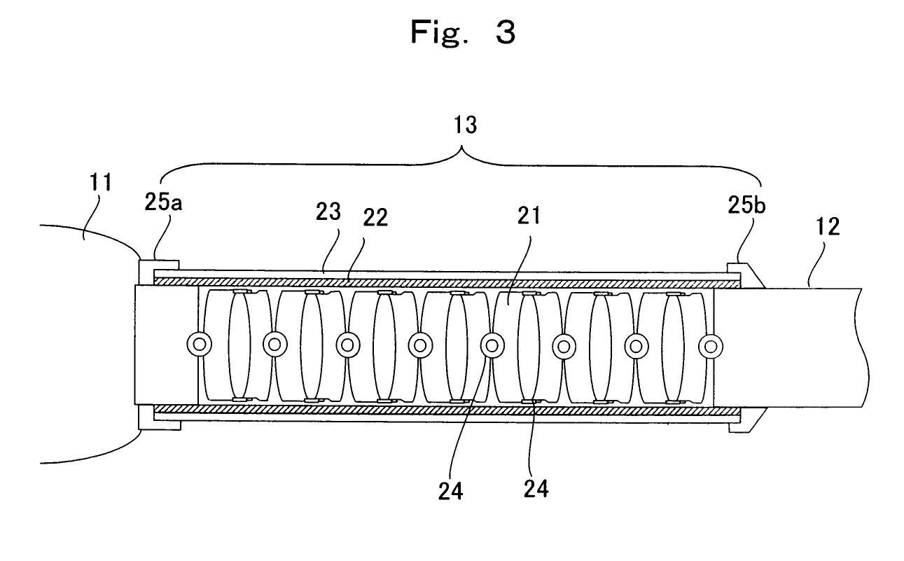

FIG. 2 is a diagram showing an internal structure of the flection of the TEE probe according to an embodiment of the present invention and the internal structure of the flection 13 is shown in such a way that the internal structure becomes evident by removing a portion of the angle tube from the state in FIG. 1. FIG. 3 shows a sectional structure of the flection 13.

As shown in FIGS. 2 and 3, the flection 13 has angle links 21 in an annular shape coated with two angle tubes 22 and 23 on an outer circumference thereof. The angle tube 22 on the inner side is electrically-conductive and the angle tube 23 on the outer side is insulating.

The flection 13 connects the guiding unit 12 and the tip unit 11 by a plurality of the angle links 21. The angle links are axis-connected by a knot ring 24 of each and can rotate around the axis up to a fixed angle determined for each. By adopting such a configuration, the tip unit 11 can be arranged at an angle with respect to the guiding unit 12 while drawing a curve. Further, the knot ring 24 is also arranged at adjacent 90 degrees rotated positions and thus, the tip unit 11 can also be arranged at an angle with respect to a directional axis perpendicular to the axis. In this manner, the angle links constitute a conductor that is curved and extensible.

The angle tubes 22 and 23 are coated to cover the angle links 21. Both ends of the angle tubes 22 and 23 are fixed to the guiding unit 12 and the tip unit 11 by fixtures 25a and 25b respectively. Accordingly, internal airtightness can be ensured to prevent a liquid such as a body fluid from penetrating into the mechanism.

Further, the angle tubes 22 and 23 have flexibility and thus, the flection 13 can be brought to a bent state accompanying a wire operation of the angle links 21. Common technology of an endoscope is used as a means for bending the flection 13. A wire (not shown) connected to the knot ring 24 is arranged inside the guiding unit 12 and the flection 13 and the flection 13 can be bent in any direction by operating the wire.

A TEE probe may be bitten by the patient while being used or the angle tube may be damaged due to an external force while being handled or ageing. If the angle tube is fractured and a hole is cut, a problem of the inner mechanism being affected by a liquid breaking in or of becoming a source of infection after bacteria being propagated in the hole may arise.

Thus, a mechanism capable of detecting insulation between water around the tip unit 11 and metal inside the probe by soaking the tip unit 11 and the flection 13 in the inspection before using the TEE probe is used.

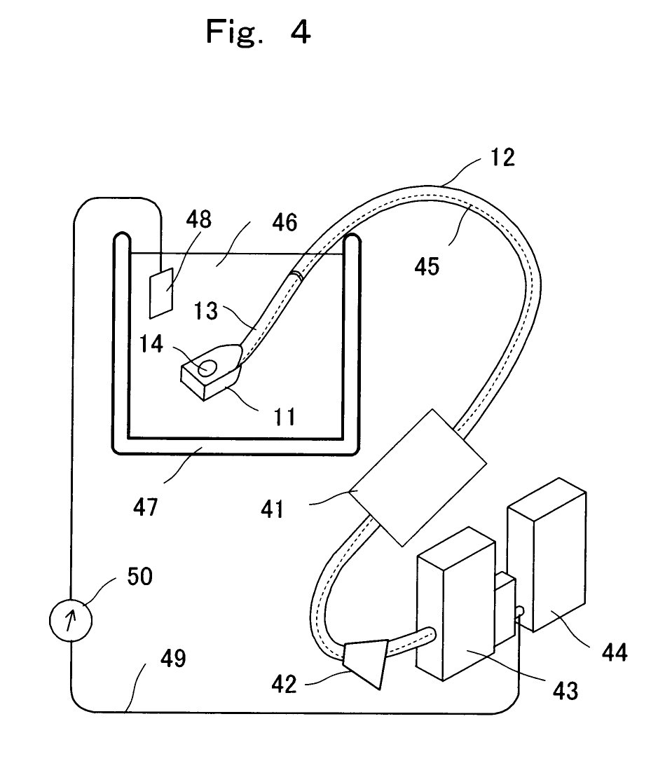

Before describing an operation to detect presence/absence of a hole in the outer angle tube 23, an overall configuration of the TEE probe shown in FIG. 4 will be described. The TEE probe includes, as described above, the tip unit 11 introduced into the esophagus, the flection 13, and the guiding unit 12 and also includes an air connector 41 provided at some midpoint of the guiding unit and containing a pump to send a liquid or an air to the tip unit 11 in the manufacturing process, a bending operation unit 42 that performs a bending operation of the flection 13, a connector unit 43, a signal transmission/reception unit 44 that transmits a driving signal to the connector unit 43 and the ultrasonic transducers 14 via the connector unit 43 and receives an electric signal converted from a wave received by the ultrasonic transducers 14, and an electric signal line 45 connected to the signal transmission/reception unit 44 and the ultrasonic transducers 14. With one electrode grounded, the ultrasonic transducers 14 vibrate due to a driving signal applied to the other electrode and transmitted from the signal transmission/reception unit 44. Normally, a liquid or a gas is emitted from the tip unit 11.

In FIG. 4, the electric signal line 45 is shown by a dotted line. Though the electric signal line 45 is shown in FIG. 4 as a curve, the electric signal line 45 is actually constituted of, for example, a wire inside the flection 13 and the guiding unit 12 and provided in a spiral shape. Moreover, instead of one line, the electric signal line 45 is composed of a plurality of lines such as two drive controlling lines to drive the ultrasonic transducers and a received signal transmission line that transmits a received signal from the ultrasonic transducers. Therefore, the TEE probe constitutes an intracorporeal monitoring apparatus having a flection.

An operator operates the bending operation unit 42 to manipulate the bending direction of the flection 13 and the angle thereof. The orientation of the tip unit can be changed by changing the direction of the flection 13. Accordingly, an ultrasonic wave transmitted/received by the ultrasonic transducers 14 provided in the tip unit 11 can be oriented in a desired direction.

FIG. 4 shows an example of the configuration to detect presence/absence of a hole in the outer angle tube 23 of the flection 13.

The tip unit 11, the flection 13, and a portion of the guiding unit 12 of a TEE probe are soaked in a liquid bath 47 filled with a physiological saline solution 46.

An electrode 48 is soaked in the physiological saline solution 46 of the liquid bath 47. A ground-side line of the drive controlling lines leading to the connector 43 and driving the ultrasonic transducers and a lead wire 49 are connected to the electrode 48 and an ammeter 50 is connected to some midpoint of the lead wire 49 to apply a high voltage.

The outer angle tube 23 is insulating and if the outer angle tube 23 functions normally, almost no current flows through the ammeter. If the angle tube 23 has a hole, the needle of the ammeter 50 jumps, which indicates that the outer angle tube 23 is not insulated.

If the presence of a hole in the outer angle tube 23 is detected, it is necessary to replace the angle tube. In contrast to an endoscope, a TEE probe secures a wide surface that emits an ultrasonic wave of the ultrasonic transducers to obtain an excellent ultrasonic image so that in most cases the diameter thereof is larger in the tip unit 11 than in the guiding unit 12.

This is because with a wider ultrasonic emitting surface, the signal to noise ratio (S/N) becomes better and the focus can advantageously be reduced in terms of resolution.

An angle tube has almost the same diameter as the guiding unit 12 and it is necessary for the angle tube to pass through the tip unit 11 having a larger diameter for replacement. While the angle tube has stretching properties necessary for bending, fluorubber is frequently used as a material thereof and has its limit to stretchability. Thus, if the angle tube is made thicker, the overall stretch amount decreases so that the angle tube may not be replaceable beyond the tip unit 11.

Flexing resistance properties are demanded for a TEE probe having no optical system so that the TEE probe is not abruptly bent in the throat or esophagus during insertion. Therefore, an optimal thickness is calculated from the elastic constant of the rubber used for the angle tube. However, the angle tube cannot be made to have an optimal thickness due to a problem of stretchability for replacement so that there is a tradeoff relation between ultrasonic image performance and flexing resistance properties.

Thus, in the present embodiment, a configuration of coating the flection 13 doubly with the angle tubes 22 and 23 is adopted. In this manner, each angle tube is made easier to stretch and also easier to replace. Then, the elastic constant of rubber can be made an optimal value of flexing resistance properties with the whole rubber of two angle tubes.

Further, while the outer angle tube 23 (outermost layer) uses non-conductive rubber such as fluorubber like a conventional TEE probe, the inner angle tube 22 (inner layer) is configured to have conductivity.

Angle tube materials having conductivity include a material obtained by mixing a conductive filler with silicon tube as a base material. Silicon rubber is a material superior in biocompatibility and also rich in stretchability while ensuring safety of a living body, making silicon rubber a suitable material for the angle rubber 22. The inner angle tube 22 having conductivity and the angle links 21 are electrically connected. Accordingly, if a hole is cut in the outer angle tube 23, a fracture of the outer angle tube 23 can be detected by the above inspection.

If the degree of fracture is severe and a hole reaches the angle 2, a fracture of the outer angle tube 23 can be detected by a similar inspection. However, whether only the outer angle tube 23 is fractured or both the angle tubes 22 and 23 are fractured cannot be distinguished and thus, it is desirable to replace both the angle tubes 22 and 23 when an angle tube fracture is detected.

By adopting a double structure of the outermost layer and the inner layer of angle rubber in the present embodiment, as described above, the thickness of the whole angle tube can be made a thickness having the optimal flexing resistance properties so that the flexing resistance properties can be improved. Moreover, the thickness of each angle tube can be made thinner than a conventional angle tube and thus, the angle tube can be replaced without changing the size of the tip unit. Therefore, excellent ultrasonic images can be obtained without sacrificing obtained ultrasonic images.

In the present embodiment, a case of one inner layer, that is, an angle tube of a two-layer structure as a whole is described. In some cases, it is advantageous to design two inner layers or more, instead of one later. That is, after the size of the tip unit 11 that decides ultrasonic image performance and the thickness of the whole angle tube of the flection 13 so as to have optimal flexing resistance properties being independently designed, the number of layers of the angle tube is decided so that the angle tube can be replaced beyond the tip unit 11.

In such a case, the angle tube in the outermost layer is made insulating and other inner layers conductive. Accordingly, a TEE probe can be made an F-type applied portion.

According to a TEE probe in the present invention, the angle tube can be inspected by a method similar to a conventional one and further, a tip unit of the conventional size can be used so that no image quality is sacrificed. Moreover, flexing resistance properties of the flection can be improved without affecting durability or costs of a mechanism unit.

In the above embodiment, a case where a structure having a flection of the present invention is applied to a TEE probe is described. However, the present invention can be applied not only to a TEE probe, but also to a laparoscope or endoscope having a flection.

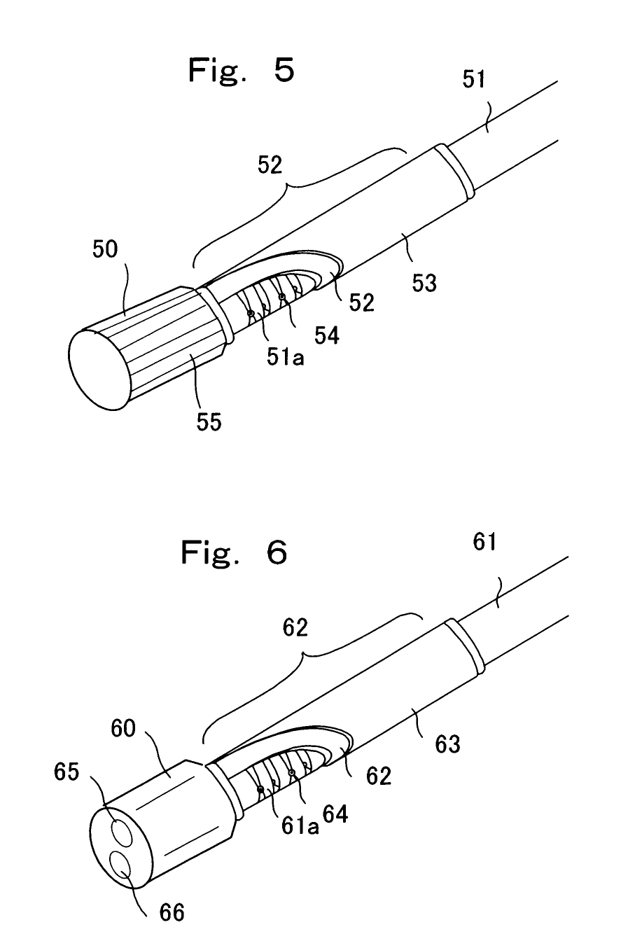

FIG. 5 shows a perspective view when an embodiment of the present invention is applied to an ultrasonic laparoscope. This perspective view shows a notch sectional view in a portion thereof. A flection 52 is provided between a tip unit 50 and a guiding unit 51 of the ultrasonic laparoscope and the flection 52 has, as shown in FIG. 2 of the above embodiment, angle links 51a coated doubly with an inner angle tube 52 and an outer angle tube 53 therewithout. The angle links 51a are axis-connected by a knot ring 54.

The tip unit 50 has a structure in which ultrasonic transducers 55 are arranged around the side surface of a cylindrical body in a longitudinal direction with respect to the central axis. These ultrasonic transducers 55 are driven by being sequentially switched by, for example, a driving signal from the signal transmission/reception unit 44 via the connector unit 43 shown in FIG. 4 and the flection 52. A reflected wave of an ultrasonic wave transmitted from a plurality of the ultrasonic transducers 55 is received again by these ultrasonic transducers 55 and processed by the signal reception unit after being converted into an electric signal and passed through the flection.

The ultrasonic laparoscope constitutes an intracorporeal monitoring apparatus having a flection.

With the configuration shown in FIG. 4, an ultrasonic laparoscope in the present embodiment can also detect presence/absence of a hole in the outer angle tube easily. That is, an ammeter is provided between a signal line passing from the tip unit through the flection and guiding unit and an electrode soaked in a liquid bath filled with a physiological saline solution and a high voltage is applied. Based on the magnitude of a current flowing through the electric signal line, presence/absence of a hole on the outer angle tube can be detected.

Angle tube materials having conductivity include a material obtained by mixing a conductive filler with silicon rubber as a base material. Silicon rubber is a material superior in biocompatibility and also rich in stretchability while ensuring safety of a living body, making silicon rubber a suitable material for the angle tube.

According to the present embodiment, an ultrasonic laparoscope whose angle tube can easily be replaced and capable of easily detecting a hole in the outer angle tube can be obtained.

Next, another embodiment when the structure of a flection of the present invention is applied to an endoscope will be described.

FIG. 6 shows a perspective view when an embodiment of the present invention is applied to an endoscope. This perspective view shows a notch sectional view in a portion of a flection 62. The flection 62 is provided between a tip unit 60 and a guiding unit 61 of the endoscope and the flection 62 has, as shown in FIG. 2 of the above embodiment, angle links 61a coated doubly with an inner angle tube 62 and an outer angle tube 63 therewithout. The angle links 61a are axis-connected by a knot ring 64.

Two optical systems are provided at the tip of the tip unit 60. One is an illuminating optical system 65 and the other an observational optical system 66. Light is shone from the tip unit 60 by the illuminating optical system 65. This light is guided by, for example, a light guide via the flection and guiding unit. The observational optical system 66 is used to receive reflected light from tissues of a subject of the light shone by the illuminating optical system.

The received light undergoes photoelectric conversion by CCD connected thereafter before being transmitted as an electric signal to the signal transmission/reception unit as shown, for example, in FIG. 4 via the flection and guiding unit.

A liquid or a gas may be emitted from the tip unit 60. The endoscope constitutes an intracorporeal monitoring apparatus having a flection. Angle tube materials having conductivity include a material obtained by mixing a conductive filler with silicon rubber as a base material. Silicon rubber is a material superior in biocompatibility and also rich in stretchability while ensuring safety of a living body, making silicon rubber a suitable material for the angle rubber 22.

With the configuration shown in FIG. 4, an endoscope in the present embodiment can also detect presence/absence of a hole in the outer angle tube easily. That is, an ammeter is provided between a signal line passing from the tip unit through the flection and guiding unit and an electrode soaked in a liquid bath filled with a physiological saline solution and a high voltage is applied. Based on the magnitude of a current flowing through the electric signal line, presence/absence of a hole on the outer angle tube can be detected. In the case of the endoscope, a dedicated valve (air connector) to send the air to the tip unit is in most cases provided.

In the above embodiments, a laparoscope using an ultrasonic wave and an endoscope using optical systems have been described. However, if the above structure of a flection is provided, the present invention can also be applied to a laparoscope using optical systems or an endoscope using an ultrasonic wave and thus, such embodiments are also included in the scope of the present invention.

Conventionally, an endoscope detects a tube break or a pinhole based on a change in pressure after pneumatic pressure being applied from a control unit. However, a TEE probe normally does not have a means for detecting air leakage. In the present invention, insulation properties of the outermost tube are electrically detected without applying pneumatic pressure. Therefore, according to detection of insulation shown in FIG. 4, when compared with detection by applying pneumatic pressure, there is an advantage of a shorter measuring time.

Conductive tubes are used for inner tubes and a break of only the outer layer can also be detected by measurement of electric insulation resistance so that a break of the outermost layer can be detected by measurement of insulation resistance as a product structure.

The present invention is not limited to the above embodiments only and can be embodied by modifying components thereof without deviating from the scope thereof when the present invention is carried out. Various inventions can be formed by appropriately combining a plurality of components disclosed in the above embodiments.

For example, some components may be deleted from all components shown in an embodiment. Further, components common to different embodiments may appropriately be combined. These modifications are also included in the present invention as long as technical ideas of the present invention are used.

What is claimed is:

1. An intracorporeal monitoring apparatus having a flection, comprising:
a tip unit that receives an ultrasonic signal or light from inside a subject and converts the ultrasonic signal or light into an electric signal;
a guiding unit that transmits the electric signal obtained by the tip unit; and
a flection that is provided between the guiding unit and the tip unit, transmits the electric signal obtained by the tip unit to the guiding unit, and can be bent, the flection has a conductor that is curved and extensible, one or a plurality of layers of conductive tubes coating the conductor, and a layer of an insulating tube coating the conductive tubes.

2. The intracorporeal monitoring apparatus having a flection according to claim 1, wherein the tip unit transmits the ultrasonic signal into the subject and receives a reflected signal thereof.

3. The intracorporeal monitoring apparatus having a flection according to claim 2, wherein the conductive tubes are constituted of silicon rubber mixed with a conductive filler.

4. The intracorporeal monitoring apparatus having a flection according to claim 3, wherein the conductive tubes are constituted of the plurality of layers.

5. The intracorporeal monitoring apparatus having a flection according to claim 1, wherein the tip unit shines the light into the subject and receives reflected light thereof.

6. The intracorporeal monitoring apparatus having a flection according to claim 1, wherein the conductive tubes are constituted of silicon rubber mixed with a conductive filler.

7. The intracorporeal monitoring apparatus having a flection according to claim 6, wherein the conductive tubes are constituted of the plurality of layers.

8. An intracorporeal monitoring apparatus having a flection, comprising:
a tip unit that receives an ultrasonic signal or light from inside a subject and converts the ultrasonic signal or light into an electric signal;
a guiding unit that transmits the electric signal obtained by the tip unit; and
a flection that is provided between the guiding unit and the tip unit, transmits the electric signal obtained by the tip unit to the guiding unit, and can be bent, the flection has a conductor that is curved and extensible, one or a plurality of layers of conductive tubes coating the conductor, and a layer of an insulating tube coating the conductive tubes, wherein the conductor that is curved and extensible is constituted of circular angle links connected by a knot ring.

9. An intracorporeal monitoring apparatus having a flection, comprising: a tip unit that receives an ultrasonic signal or light from inside a subject and converts the ultrasonic signal or light into an electric signal; a guiding unit that transmits the electric signal obtained by the tip unit; and a flection that is provided between the guiding unit and the tip unit, transmits the electric signal obtained by the tip unit to the guiding unit, and can be bent, the flection has a conductor that is curved and extensible, one or a plurality of layers of conductive tubes coating the conductor, and a layer of an insulating tube coating the conductive tubes, wherein
the tip unit and the flection are soaked in water, an ammeter is installed between an electrode placed in the water and a conducting wire transmitting an electric signal of the tip unit, and insulating properties of the insulating tube are examined by applying a high voltage to a path thereof.

10. A transesophageal echocardiography probe, comprising:
a tip unit that transmits/receives an ultrasonic wave;
a guiding unit that guides the tip unit into an esophagus; and
a flection that is connected between the tip unit and the guiding unit and can be bent by changing a connection angle between the tip unit and the guiding unit, the flection is coated with a plurality of layers of tube, an outermost tube has insulating properties, and other inner tubes are constituted of a material having conductivity.

11. The transesophageal echocardiography probe according to claim 10, wherein the inner tubes are constituted of silicon rubber mixed with a conductive filler.

12. The transesophageal echocardiography probe according to claim 11, wherein the outermost tube is constituted of a fluoresin.

13. The transesophageal echocardiography probe according to claim 12, wherein the conductive tubes are constituted of the plurality of layers.
